(19) United States
(12) Patent Application Publication
(10) Pub. No.: US 2011/0321181 A1
(43) Pub. Date: Dec. 29, 2011

(54) DEVICE TO BE PLACED IN BLOOD VESSEL, ANGIOSTENOSIS MODEL USING SAME AND METHOD FOR MAKING MODEL

(75) Inventors: Kenichi SHIMURA, Ashigarakami-gun (JP); Taizou IWASAKI, Ashigarakami-gun (JP); Fumiaki IKENO, Menlo Park, CA (US)

(73) Assignee: TERUMO KABUSHIKI KAISHA, Shibuya-ku (JP)

(21) Appl. No.: 13/224,509

(22) Filed: Sep. 2, 2011

Related U.S. Application Data
(63) Continuation of application No. PCT/JP2010/050150, filed on Jan. 8, 2010.

(30) Foreign Application Priority Data
Mar. 6, 2009 (JP) 2009-053816

(51) Int. Cl. A01K 67/00; A61L 27/30

(52) U.S. Cl. 800/9; 623/1.46

(57) ABSTRACT

An indwelling vascular device is constructed such that a vascular stenosis model can be controlled from the partial stenosis to the total occlusion. There is also described a vascular stenosis model of such non-human animal, and a method for making the same. The model can be used for diagnosis or therapy of a disease resulting from the stenosis or total occlusion in a blood vessel or further for the development of therapeutic approaches. The indwelling vascular device includes a device substrate containing a metal and/or metal compound which elutes toxic metal ions at least from a surface thereof and having a structure ensuring a vascular flow immediately after indwelling in a blood vessel, and a polymer coating layer formed on at least a metal and/or metal compound containing surface of the substrate.

This application is a continuation of International Application No. PCT/JP2010/050150 filed on Jan. 8, 2010, and claims priority to Japanese Application No. 2009-053816 filed on Mar. 6, 2009, the entire content of both of which is incorporated herein by reference.

### TECHNICAL FIELD

The disclosure here relates to an indwelling vascular device for making a vascular stenosis model in animals, a stenosis or total occlusion model of a blood vessel in animals using the same, and a method for making the model. The disclosure also involves the use of the model for diagnosis or therapy of a disease resulting from the stenosis or total occlusion in a blood vessel or further for the development of therapeutic approaches.

### BACKGROUND DISCUSSION

According to the vital statistics of the Ministry of Health, Labour and Welfare, in 2006, deaths from heart disease, which is a main disease of the circulatory system, are the second cause of death domestically. Ischemic heart disease, based mainly on myocardial infarction, occupies a major proportion of these deaths. A therapeutic method used for ischemic heart disease is the PCI (percutaneous coronary intervention) technique in which a guide wire is fed to a narrowed area of a blood vessel and the narrowed area of the blood vessel is then dilated. This PCI technique has relatively good success rate, owing to the development of medical devices and the improvement in therapeutic techniques, and the success rate with respect to a pathological lesion not totally occluded exceeds 95% at the moment. However, with a totally occluded lesion, i.e. so-called chronic total occlusion (CTO), the tissue undergoes calcification or a high degree of fibrosis and a difficulty occurs in the passage of a guide wide in most cases. Thus, the success rate has been at about 70% and the improvement after 1990 has been only slight at the present as discussed in Non-Patent Documents 1 to 3 identified below. Hence, there has been required a heart disease model useful for the development of a therapeutic method in a chromic total occlusion lesion and also for a training procedure, especially for the training of a guide wire procedure.

The accumulated clinical data points out that for the development of myocardial infarction, stenosis and occlusion of coronary arteries caused by the neointimal proliferation, blood clot or the like occurring at the lesion area of arteriosclerosis, play an important role. In association with such a circulatory system disease or organ transplantation, validation making use of laboratory animals or animal models has to be made, like other diseases, so as to study and establish therapeutic methods and carry out developments such as of medicines, medical devices and the like.

Conventionally, heart disease models in a variety of animals such as rodents (rat, mouse, rabbit), dogs, pigs or primates have been proposed. These have been made such as by endothelial desquamation caused by scratching (see Non-Patent Document 4 identified below), overdilatation of blood vessel (see Non-Patent Documents 4 to 5 identified below), electric stimulation (see Non-Patent Document 6 identified below), heat (see Non-Patent Document 7 identified below), exposure to air (see Non-Patent Document 8 identified below), cholesterol loading (see Patent Document 1 and Non-Patent Document 9 identified below) and others.

In the current heart disease model methods set out in this literature, not only is the stenosis rate difficult to control, but so too it is difficult to obtain stable models. Especially, the cholesterol loading model has a problem in that it takes a long time before ready for use along with a high-cost load.

Several methods of making a total occlusion model have been reported. For instance, a technique of embolizing a blood vessel by insertion of a blood clot or a clotting factor thereinto, for example, according to a surgical procedure is shown. However, such a surgical procedure has a problem in that an invasiveness against animals is great (see Patent Documents 2 and 3 identified below).

Suzuki et al have reported a total occlusion model making use of a technique wherein bone pieces and a gelatin sponge are mixed and inserted into a coronary artery by intervention (see Non-Patent Document 10 identified below). Although the surgical invasiveness can be suppressed according to this method, a survival rate is as low as 60% and the total occlusion forming rate is at 50%, thus being unsatisfactory for practical applications.

It has been reported that when a copper stent that allows copper ions to be eluted is indwelled in a coronary artery, neointimal proliferation is observed (see Non-Patent Documents 11 to 13 identified below). Because the action of electrothrombosis of copper at an acute phase (see Non-Patent Document 14 identified below) or intimal damages ascribed to the chemical toxicity of copper itself (see Non-Patent Documents 15 to 16 identified below) are brought about thereby quickly forming a clot, the indwelling of a copper stent in the coronary artery has the very high possibility of resulting in death within a very short time.

### Patent Documents

Patent Document 1: Japanese Patent Laid-Open No. 2004-89148
Patent Document 2: JP-T-2005-503820
Patent Document 3: WO 2006/030737

### Non-Patent Documents

Non-Patent Document 1: J Eur Heart J 2005; 26:2630-6
Non-Patent Document 2: J Am Coll Cardiol 2001; 38:409-14
Non-Patent Document 3: J Am Coll Cardiol 2007; 49:1611-8
Non-Patent Document 4: Circulation 2000; 101:812-8
Non-Patent Document 5: Arterioscler Thiomb Vasc Biol 1994; 14:395-400
Non-Patent Document 6: Am J Pathol 1997; 150:761-76
Non-Patent Document 7: Circulation 1992; 86:1249-56
Non-Patent Document 8: J Vasc Res 1996; 33:308-14
Non-Patent Document 9: J Card Surg 2008; 23:312-20
Non-Patent Document 10: J Invasive Cardiol 2008; 20:296-301
Non-Patent Document 11: Nippon Acta Radiologica 1991; 51:1195-20
Non-Patent Document 12: J Invasive Cardiol 1997; 10:61-9
Non-Patent Document 13: J Invasive Cardiol 2005; 17:452-4
Non-Patent Document 14: J Neurosurg 1975; 42:76-85
Non-Patent Document 15: J Neurosurg 1989; 70:751-4
Non-Patent Document 16: Japanese Journal of Clinical Ophthalmology 1989; 43:1349-52

### SUMMARY

As stated above, under the circumstances where vascular stenosis and total occlusion models effective for procedural training for the treatment of the stenosis or total occlusion in blood vessels and also for the development of a novel therapeutic device are demanded. The disclosure here provides a coronary artery stenosis or total occlusion model in a stable pathological condition where a variation in degree of stenosis is relatively small. The disclosure here also provides a method for making such a model that is applicable, without limitation, to any blood vessels such as of the right coronary artery, left anterior descending artery, left circumflex artery and the like at a low lethal rate and can be obtained within a short time even with the case of a total occlusion model and also of a device therefor. In particular, the disclosure provides a pathological condition model for vascular stenosis and total occlusion, which helps ensure a high survival rate in animals having a blood stream and vessel size close to humans.

The inventors here have made intensive studies on the above problems and found that when the surface of a indwelling vascular device including a material capable of eluting toxic metal ions having the electrothrombotic action and chemical toxicity is coated with a polymer film capable of appropriately controlling the elution of the metal ions, there can be made, at a high survival rate, a vascular stenosis model that is controlled in the degree of the stenosis. Especially, it has been confirmed that animals indwelled with the polymer film-formed device become a total occlusion pathological condition model in about one week and remain viable over at least four weeks. On the other hand, animals indwelled with a device having no such a film and capable of freely eluting metal ions have died within a short time. When determined according to JIS T0304, the total elution amounts (in one week) of the metal ions from the thin polymer film-coated device and a polymer film-free device have been found to be substantially equal to each other irrespective of the presence or absence of the polymer film. Moreover, it has also been found that when comparing the total elution amounts of the metal ions from the thin polymer film-coated device and the polymer film-free device within short times (of 0 to 168 hours), an initial elution amount of the polymer film-coated device becomes significantly lessened. Accordingly, with a device provided with the polymer film, it is suggested that initial thrombus formation is inhibited, so that occlusion develops slowly thereby mitigating a load on animal and that direct contact between the living tissue and the copper per se is avoided and thus, intimal damages resulting from the chemical toxicity of copper itself are mitigated, thereby enabling a total occlusion pathological condition model to be made without death.

It has been confirmed that with a device of the type wherein the polymer film is provided within a certain range of thickness, the film thickness is in inverse relation with an elution amount of copper ions and also with a vascular stenosis rate. Hence, it has been found that if the indwelling period is kept constant, the stenosis rate including a complete occlusion (100% DS: % diameter stenosis) can be controlled by the film thickness. On the other hand, it has also been found that when the indwelling period is varied while keeping the film thickness constant, the stenosis rate can be controlled. Accordingly, the disclosure here provides the followings based on this knowledge.

An indwelling vascular device disclosed here includes a device substrate containing a metal and/or metal compound (hereinafter referred to as a metal) capable of eluting toxic metal ions at least from a surface thereof and having a structure ensuring a vascular flow immediately after indwelling in a blood vessel, and a polymer coating layer formed on at least a metal-containing surface of the substrate.

The device substrate may be one that is able to elute toxic metal ions from the substrate surface and may be accordingly such that the substrate itself may be made totally of the metal or may have a structure having the metal on a surface portion formed such as by plating.

The polymer preferably contains units derived from para-xylylene and/or a derivative thereof (which may be hereinafter called parylene).

The thickness of the polymer coating layer can vary depending on, for example, the type of polymer or a desired stenosis rate, but is generally at least 0.001 μm, preferably not less than 0.01 μm. On the other hand, a film that is too thick can completely impedes elution of toxic metal ions and so the maximum thickness of the polymer coating layer is up to 10 μm. For instance, with a parylene film, the stenosis action is shown at a level of not greater than 3 μm.

As the toxic metal ions, copper ions are of particular concern.

The elution amount of copper, determined by measurement of JIS T0304, of the device of the invention is generally at 4 to 1500 μg per device.

The device is not critical in type so far as it is a device of a vascular indwelling structure. A preferred form of the device is a stent.

The method for making a vascular stenosis model according to the disclosure here includes indwelling an indwelling vascular device as set out above in a blood vessel of a nonhuman healthy animal.

In this method, the stenosis rate can be controlled by the film thickness of the polymer film layer of the device, and a total occlusion model can be made.

Fabrication may be made within a short time of one to two weeks without limitation on employed blood vessels including not only the right coronary artery, but also the left anterior descending artery, the left circumflex artery and the like. This is true of a total occlusion model. Moreover, with respect to animals closer to human beings such as, for example, pigs and the like, fabrication of such a model can be realized.

The disclosure here also provides a non-human animal indwelled with such a device as set out above in a blood vessel.

More particularly, the non-human animal is a vascular stenosis model. The stenosis rate in this model is controllable and a total occlusion model may also be provided.

The total occlusion means a pathological condition in which the vascular stenosis rate is 100%. That is, a condition in which the vessel is completely blocked. In this specification, the total occlusion and stenosis are used to refer to complete blood vascular blockage and partial blood vascular blockage respectively, though it is to be understood that total occlusion is considered one embodiment or type of vascular stenosis (i.e., stenosis includes total occlusion as well as partial occlusions).

No limitation is placed on the type of blood vessel to be indwelled with the device. A specific example includes a coronary artery.

Although the kind of animal is not critical, a pig is preferred.

The disclosure here proposes the use as a vascular stenosis or total occlusion model of non-human animals for developing a diagnosis, a therapy and a therapeutic method associated with a stenosis or total occlusion in blood vessels. More particularly, because non-human animals survive in a pathological condition of stenosis or total occlusion, the invention can be utilized for many purposes of a procedural training approach directed to observation, study and therapy of the pathological condition of stenosis or total occlusion and further of the development of a novel therapeutic device and therapeutic method along with the confirmation of the effectiveness thereof. For instance, the disclosure is useful for a guide wire procedure, especially as a procedural training model in coronary artery.

In particular, because of the survival model, the device/model/method is useful for evaluating cardiopulmonary function in association with the pathological condition of vascular stenosis or total occlusion or as a cardiopulmonary observation model. Additionally, the device/model/method is useful as a regeneration therapy model of cardiac infarct, thereby enabling the effectiveness of therapy by application of a regeneration sheet to be confirmed.

The indwelling vascular device is quite simple in structure, can be provided inexpensively, and is relatively low in invasiveness to animals. The device can be indwelled like existing stents, so that non-human animals serving as a vascular stenosis model can be readily obtained without resorting to any specific indwelling technique. Moreover, when using the device, there can be reproducibly made a total occlusion or vascular stenosis model in a stable pathological condition, which is reduced in variation at a desired stenosis rate and is low in lethal rate, within a short time of one to two weeks.

The non-human animal serving as a vascular stenosis model according to the disclosure here is applicable to a procedure for therapy of a total occlusion or stenosis in blood vessels, especially, to the development of training and therapeutic devices of coronary arteries.

The disclosed total occlusion model or stenosis model is very useful as a surviving animal model for the research and development of therapeutic training and therapeutic devices of chronic total occlusion because no acute arterial occlusion ascribed to initial clot formation is developed, so that a fatal outcome can be avoided owing to acute cardiac infarction, i.e., ischemia.

### BRIEF DESCRIPTION OF THE DRAWING FIGURES

FIG. 1 schematically shows a vapor deposition process of parylene.

FIG. 2(A) is an electron microscope photograph of a device surface prior to vapor deposition of parylene, and FIG. 2(B) is an electron microscope photograph of a device surface after vapor deposition of parylene.

FIGS. 3(A)-3(D) are electron microscope photographs of a surface of a parylene vapor-deposited film for different thicknesses.

FIG. 4 is a graph showing an elution amount of copper ions (one week) relative to a parylene film thickness.

FIG. 5 is a graph showing a time-dependent elution amount of copper ions relative to a parylene film.

FIGS. 6(A)-6(D) show angiographic images two weeks after indwelling and FIGS. 6(a)-6(d) show pathological images two weeks after indwelling.

FIG. 7 is a graph showing a stenosis rate in relation to a parylene film thickness (two-week indwelling).

FIG. 8 is a graph showing a stenosis rate in relation to a parylene film thickness (four-week indwelling).

FIGS. 9(A)-9(C) are pathological images of a totally occluded blood vessel stained with HE.

FIG. 10 is an angiography image two weeks after indwelling.

FIG. 11 is an angiography image four weeks after indwelling.

FIGS. 12(A)-12(C) are model angiography images of a procedure simulating the therapy of chronic total occlusion.

FIGS. 13(A) and 13(B) are cross-sectional views of an infarction area in case where indwelled areas differ from each other.

FIGS. 14(A) and 14(B) show pathological images of a coronary artery area.

FIG. 15 shows an angiography image two weeks after indwelling of a SUS 316L stent.

FIG. 16 is a pathological image in indwelling of a copper-plated stent.

FIG. 17 is a pathological image in indwelling of a copper-plated stent.

### DETAILED DESCRIPTION

The substrate of the indwelling vascular device disclosed here may have any structure so far as it is able to be retained at an intended area in a blood vessel and possesses a structure to help ensure a blood stream or blood flow immediately after indwelling in the blood vessel. According to one preferred example, the device is a stent, with which a therapeutic method and procedure have been already established clinically and which has been currently confirmed to provide pronounced results and usefulness as a device for ischemic cardiac disease.

The substrate of the device should contain metals capable of elution in the form of toxic metal ions at least from the surface of the substrate. The toxic metal ions may be metal ions, which show toxicity if present in a living body, particularly, in a blood stream, such as copper ions, lead ions, cadmium ions and the like. Typically, copper ions are mentioned. Although the metal may be in the form of a metal or metal compound capable of eluting metal ions in a blood stream, it is generally a metal or an alloy of metal. In the case where the eluted metal ions are copper ions, mention is made of copper or a copper alloy such as brass, bronze, white copper, nickel silver and the like.

The substrate itself may be made totally of such a metal as mentioned above or may be configured such that a surface portion of the device is made of the metal.

In practice, it is preferred that if the device substrate is formed so that a surface portion is made of a metal, an ordinary stent can be readily and inexpensively obtained by coverage with such a metal as mentioned above. The stent used in this fashion may be made of a material whose radiopacity may be either relatively high or relatively low. Specific examples of the material having high radiopacity include a stainless steel, tantalum or its alloy, platinum or its alloy, gold or its alloy, a cobalt-based alloy, a cobalt-chromium alloy, a titanium alloy, a niobium alloy and the like. Corrosion-resistant SUS 316L is preferred as a stainless steel.

With a stent of a material whose radiopacity is relatively low, such as a biodegradable stent, more difficulty is involved in identifying a stenosis area, which becomes a procedural training approach including the identification of the stenosis area. Thus, the range of application of the disclosure here for use as a vascular stenosis model may be more extended. Eventually, the quality as a vascular stenosis model becomes higher, with the possibility of establishing a vascular stenosis model or total occlusion model even for skilled doctors. More particularly, mention is made of homopolylactic acid, homopolyglycolic acid and copolymers thereof for high molecular weight polymers, and also of a material mainly composed of magnesium for metals.

The skeletal structure (i.e., the configuration) of the stents and the method of making the stents can be in accordance with known techniques and are not thus described in detail. For instance, stent shaping can be carried by removing, from a tubular body (more particularly, a metal pipe), portions other than the frame structure forming the stent. More particularly, a stent may be formed by removing unnecessary portions of a metal pipe, for example, by an etching method called photofabrication, which makes use of masking and chemicals, an electro-discharge machining method using a mold, cutting (e.g. mechanical polishing, laser cutting) and the like. After fabrication of the frame structure, the structure is preferably polished at its edges by chemical polishing or electrolytic polishing.

In order to permit metal to exist on the surface portion of the stent, a layer of the metal is usually formed by electroless plating, electrolytic plating, dry plating, molten metal plating, physical vapor deposition, chemical vapor deposition, wet process, chemical conversion coating, anodising ion implantation or the like. Of these, electroless plating is preferred because of the appropriate cost, the ease in operation and the high accuracy. An example of the steps ordinarily carried out as electroless plating generally includes (1) defatting step, (2) oxide film-removing step wherein an article to be plated is immersed in an acid solution, (3) electroless plating step, and (4) drying step.

Where the above-mentioned metal is provided only on a surface portion, as opposed to the entire device being made of the metal, the thickness of the metal surface portion is not critical and may generally be not less than 1 μm.

The device disclosed here has a polymer film layer at least on the metal-containing surface of the device substrate containing such a metal as mentioned hereinbefore, preferably on the entire surface of the substrate. The film layer is a polymer film that appropriately controls metal ions eluted from the device substrate. Any type of polymer materials may be used provided that they exhibit the metal ion eluting control function as set forth above, and are applicable to a living body without impeding the structure and function of the device substrate. The coating method may be appropriately selected from known methods, depending on the type of polymer material used. Preferably, the polymer film layer is formed by a chemical vapor deposition method (CVD) to help ensure the formation of a uniform film over the entire substrate surface. Especially, a preferred material is one which is able to be deposited at about room temperature in a vacuum deposition temperature and is thus applicable to a substrate not resistant to heat. Such preferred materials include, for example, parylene, polytetrafluoroethylene, polyimides, polyurea copolymers, polyacrylic esters, polypeptides and the like.

Parylene is a generic term for para-xylylene and derivatives thereof, for which mention is made of parylene N (para-xylylene) whose aromatic ring is free of a functional group, parylene C wherein one of the hydrogen atoms of the aromatic ring is replaced by chlorine, parylene M wherein one hydrogen is replaced by methyl group, and parylene F wherein one of the methylene groups is fluorinated. The respective units derived from these are indicated below.

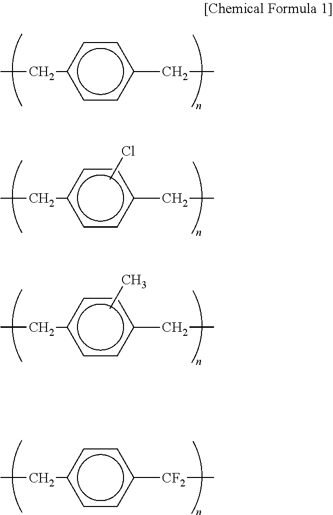

### Parylene N Parylene C Parylene M Parylene F

There are known derivatives improved in heat resistance, derivatives having fluorescent characteristics and the like, which are usable in the invention. In addition, copolymers of parylene and other type of compound may also be used. Among them, parylenes N and C satisfy biological requirements of ISO 10993 and USP analysis IV plastics, and have been registered in the Device Master File and Drug Master File of FDA and are applicable to the medical field.

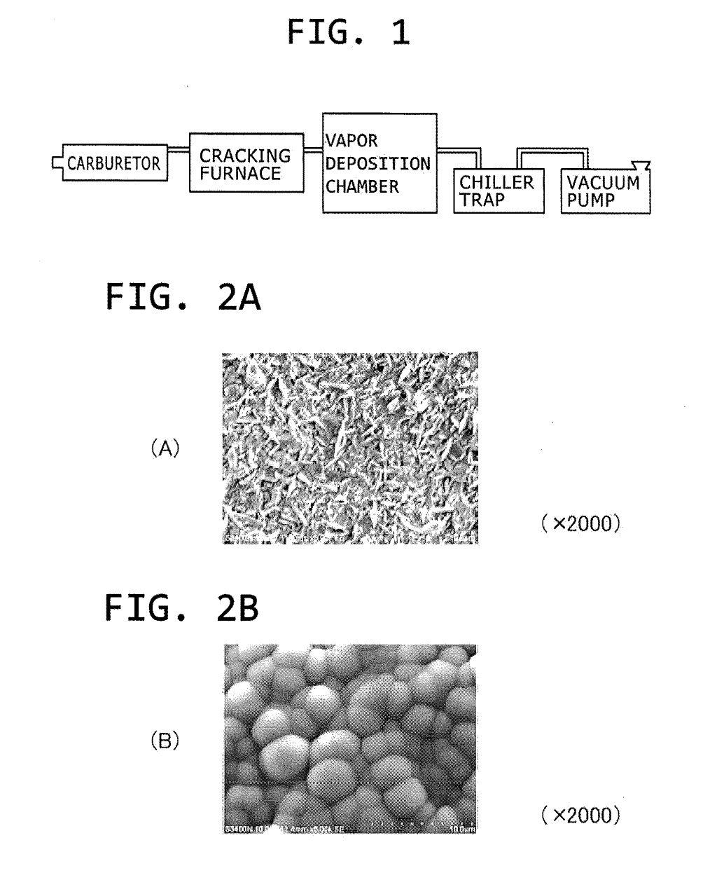

For the formation of a polymer film layer from the parylene, a CVD method is generally applied. The CVD methods per se of parylene are known and the formation may be carried out according to these methods. In general, a process shown in FIG. 1 is carried out. More particularly, an apparatus is initially reduced to a pressure of about 1 to 4 Pa by a vacuum pump, 1) a dimer placed in a carburetor is heated to a temperature of 100 to 180° C. and sublimated, 2) the dimer is passed through a cracking furnace of 650 to 700° C. for conversion into a monomer gas, 3) followed by polymerization in a vapor deposition chamber to deposit the resulting parylene on a substrate surface to thereby form a coating layer. The chemical structures of the parylene dimer, monomer and polymer at the respective stages of 1) to 3) above are indicated below with respect to parylene N.

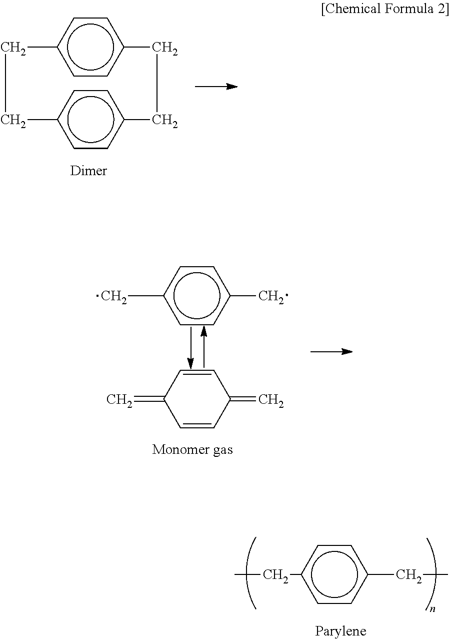

In an embodiment disclosed here wherein a polymer film layer made of such parylene as indicated above is formed, for example, on a copper-plated surface of a SUS substrate, such a structure is observed that, as shown in FIG. 2, the copper deposits (see FIG. 2(A)) on the SUS surface, on which the parylene is placed/coated (see FIG. 2(B)). When the device disclosed here is indwelled in a blood vessel, the amount of the toxic metal ion eluted from the substrate surface can be controlled by the polymer film layer. As stated hereinbefore, with respect to the elution amount of the metal ions, a measurement of the elution test JIS T0304 for an ordinary metal-based biomaterial (i.e. a cumulative value over one week) may not always give an indication, but provides an indication for the control of an elution amount with the polymer film layer during a given period of time. Accordingly, although limitation is not placed on the elution amount determined according to JIS T0304, the elution amount based on JIS T0304 is preferably 4 μg to 1500 μg per device, more preferably 4 μg to 1000 μg, for example, with respect to copper ions. In practice, it has been confirmed that when the elution amount of copper ions is so controlled as indicated above, partial stenosis to total occlusion can be controlled accurately within a relatively short time. If the elution amount is less than 4 μg, intimal damages ascribed to the thrombotic action induced by electrocoagulation of copper and the chemical toxicity of copper per se are not fairly observed and not only total occlusion, but also stenosis is observed only slightly although depending on the individual difference of non-human healthy animals. In case stenosis is observed, it takes a relatively long time and a difficulty is involved in accurately controlling the stenosis, thereby increasing a costly load. On the other hand, if the elution amount is over 1500 μg, the thrombotic action based on the electrocoagulation abruptly appears. In some cases, death occurs owing to acute thrombotic total occlusion. Thus, because of the lowering of survival rate and the increase of a costly load, the use as a stenosis or total occlusion model is unlikely to be provided. That is, if the elution amount is less than 4 μg or more than 1500 μg, the disclosure here may not be as well suited to being used as a stenosis model, including a total occlusion model.

The thickness of the polymer film layer is in inverse relation with the elution amount of JIS T0304 and also with a stenosis rate. Accordingly, the stenosis rate can be controlled by the thickness of the polymer film layer. A preferred thickness of the polymer film layer may differ depending on, for example, the type of polymer and also on a desired stenosis rate. The thickness is generally at least 0.001 μm, preferably not less than 0.01 μm. On the other hand, an excessively large thickness completely impedes the elution of toxic metal ions. The maximum thickness is preferably not greater than 10 μm, more preferably not greater than 5 μm. For example, it has been confirmed that with the case of a parylene film, the stenosis action is shown when the thickness is not greater than 3 μm.

Parylene is commercially available and, for example, both Parylene N and Parylene C are available from Daisan Kasei Co., Ltd.

The thickness of the polymer film layer can be measured by an optical interferotype film thickness meter. The film thickness can be controlled by preliminarily obtaining a calibration curve of thickness measurements relative to the amount of a monomer material.

In practice, such a device as set out above is indwelled in a blood vessel of non-human healthy animals. Applicable non-human animals may be any animal species if they are laboratory animals and specific examples include pig, minipig, rat, mouse, rabbit, guinea pig, dog, monkey and the like. Of these, a pig that is an experimental animal suitable here is preferred because it has a number of similarities to human beings from physiological and anatomical standpoints and with respect to from a feeding habit to physiology concerning digestion absorption and in respect of blood vessels, particularly the running and endothelial structures of coronary arteries, and also from the current standpoint of animal protection.

In this method, the stenosis rate can be controlled by the thickness of the polymer film layer of the device and a total occlusion model can be made. In particular, the device can be made within a short time of one to two weeks, with no limitation being placed on the type of blood vessel to be used such as of right coronary artery, left anterior descending coronary artery or left circumflex artery. This is true of a total occlusion model. Moreover, fabrication of such a model can be realized with respect to animals, such as pig, which are closer to human beings.

Animals after indwelling of the device are ensured to have a satisfactory duration of survival after establishment of a vascular stenosis or total occlusion model. In examples, survival over at least four weeks (death by autopsy) was confirmed.

The disclosure here provides a non-human animal wherein such a device as set out hereinabove is indwelled in a blood vessel thereof. Particularly, the non-human animal can be provided as a vascular stenosis model or a total occlusion model.

### EXAMPLES

The disclosure here is particularly described by way of examples. The examples described here are merely typical ones within the scope of the disclosure here and should not be construed as limiting the invention thereto.

### Example 1

### Device Fabrication

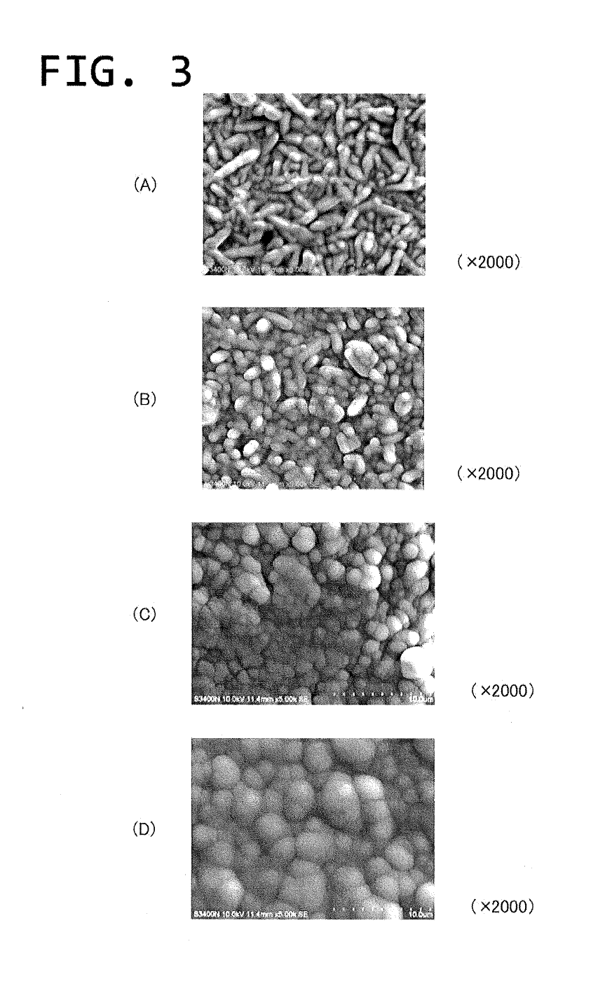

A coronary artery stent (φ3.0 mm×length 15 mm) made of SUS 316L was subjected to copper plating by an electroless plating method so that the stent has a copper plating thickness of 5 μm. Using a CVD apparatus, Parylene C (made by Daisan Kasei Co., Ltd.) was vapor deposited, in different targeted thicknesses of 0.1, 0.5, 1.0 and 3.0 μm, on three samples in each thickness while making use of an amount of the parylene based on a preliminarily prepared calibration curve (n=5 in the respective weights of the parylene). The noted thicknesses are target thicknesses. The electron micrographs of (x 2000) of the respective deposited film surfaces are shown in FIGS. 3(A)-3(D), where FIG. 3(A) shows the film surface thickness of 0.1 μm, FIG. 3(B) illustrates the film surface thickness of 0.5 μm, FIG. 3(C) shows the film surface thickness of 1.1 μm and FIG. 3(D) depicts the film surface thickness of 3.1 μm. The referenced film surface thicknesses in FIGS. 3(A)-3(D) are measured film thicknesses.

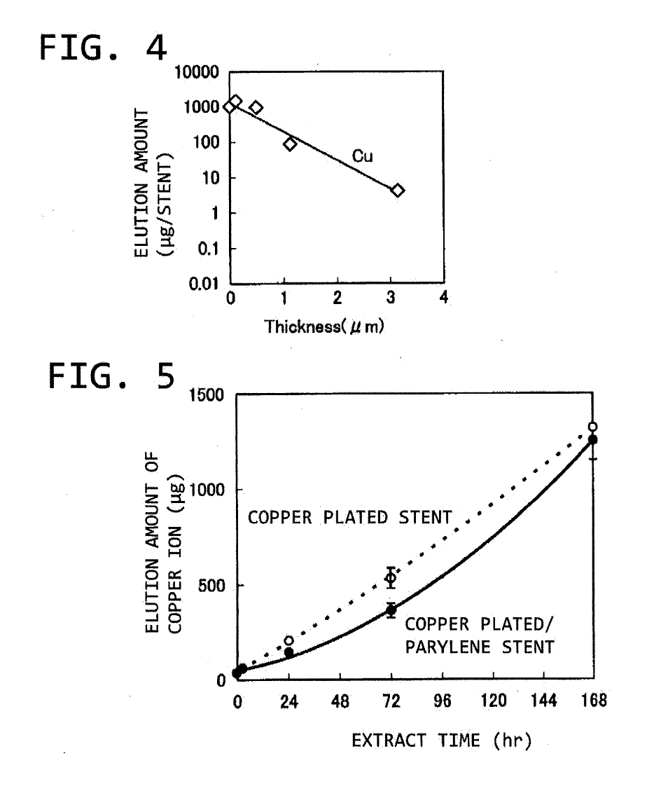

The deposited films were subjected to measurement of an elution amount of copper ions according to JIS T0304. In brief, the individual stents were immersed in a 1% lactic acid solution for one week. The relation between the parylene film thickness and the elution amount of copper ions is shown in FIG. 4. Both are in inverse relation with each other. A stent having a 0.1 μm thick parylene film and a parylene film-free stent were immersed in a similar solution to measure an elution amount (cumulative) of copper ions over a time of from 0 hours to 168 hours, with the results shown in FIG. 5. The elution amount from the parylene film-deposited stent is reduced in particular during periods of time of 24 hours to 72 hours when compared with the parylene film-free stent.

### Example 2

### Poly(para-xylylene) Film Thickness and Degree of Stenosis of Pig Coronary Artery

The deposited film-coated stent of Example 1 was crimped on a PTCA balloon and sterilized with an ethylene oxide gas. This was indwelled in the anterior descending coronary of the left coronary artery of a pig under anesthesia at a dilation ratio being 1.3 times greater. This dilation rate refers to the stent being expanded 1.3 times its original size during insertion of the stent. The pig was orally administered with antiplatelet drugs (330 mg of aspirin and 200 mg of ticlopidine) and a beta-blocking drug (10 mg of propranolol hydrochloride) from three days before the indwelling till the day before autopsy.

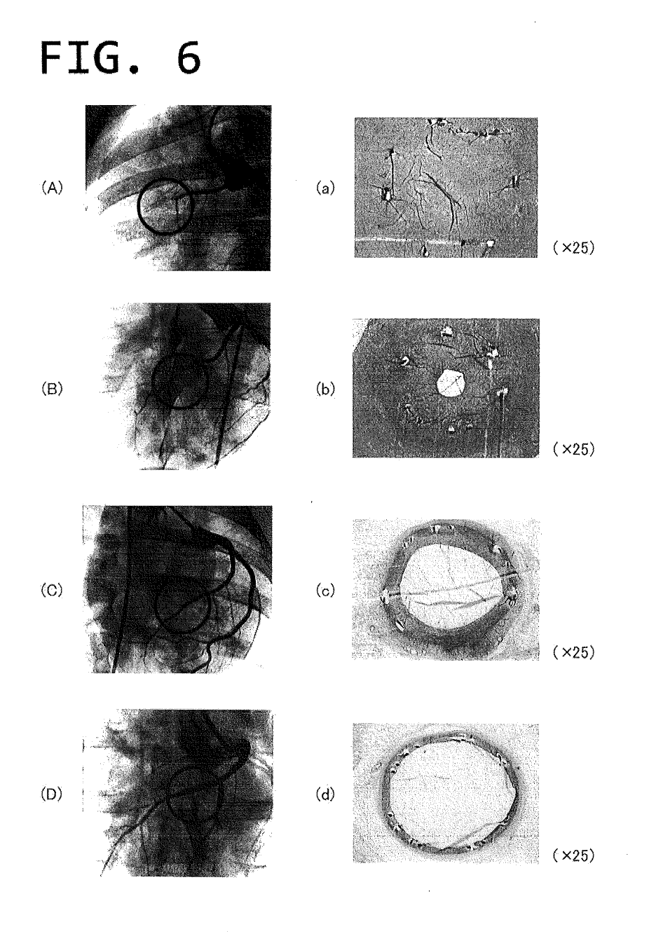

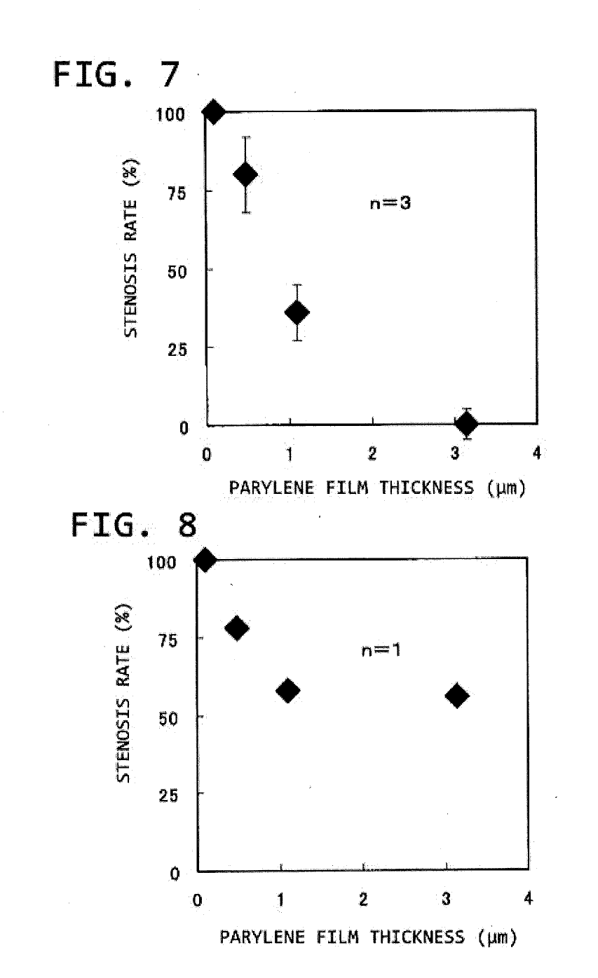

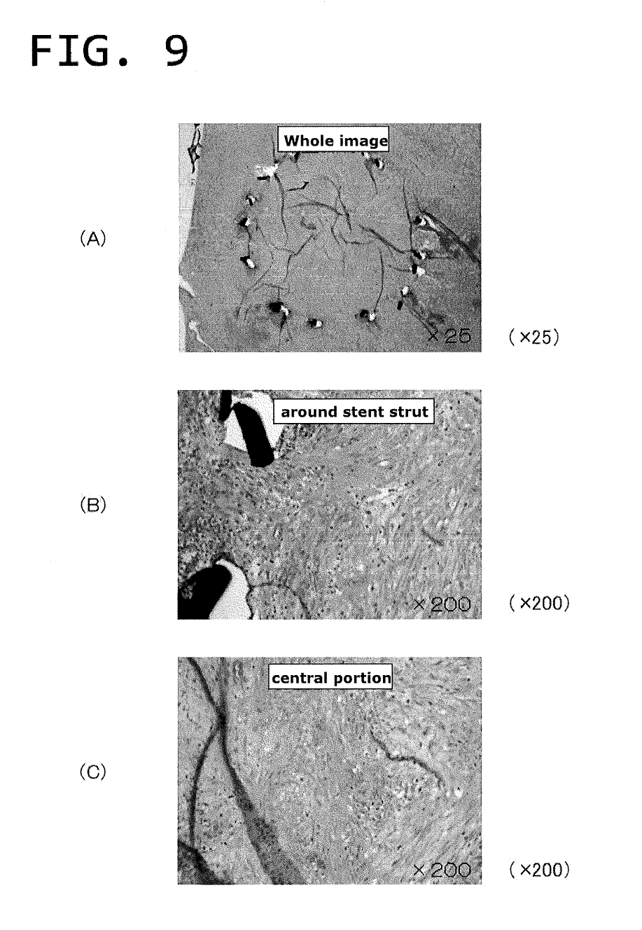

Angiography was carried out at the second week after the above treatment. Angiography images are shown in FIGS. 6(A) to (D) and pathologic images are shown in FIGS. 6(a) to (d). With respect to the respective thicknesses of the parylene C deposited film, the coronary artery indwelled with the 0.1 μm thick film-deposited stent was totally occluded (FIGS. 6(A) and (a)). With the coronary artery indwelled with a 0.5 μm thick film-deposited stent, % diameter stenosis (% DS) is at 80% (FIGS. 6(B) and (b)), with the coronary artery indwelled with a 1.1 μm thick film-deposited stent, % DS is at 36% (FIGS. 6(C) and (c)), and with coronary artery indwelled with a 3.1 μm thick film-deposited stent, % DS is at 0% (FIGS. 6(D) and (d)). FIG. 7 shows a stenosis rate relative to the parylene film thickness. The stenosis rate relative to the parylene film at the fourth week is shown in FIG. 8.

From the above results, it has been verified that when the parylene film thickness is changed, the control of the stenosis rate is possible and that the stenosis rate can also be controlled by controlling an indwelling period of time.

After undergoing euthanasia, the totally occluded blood vessel (FIGS. 6(A) and (a)) was taken out and a resin-embedded section was prepared and subjected to HE staining. The HE-stained pathologic image is shown in FIG. 9. A number of inflammatory cells and smooth muscle and fibroblast cells are observed about the stent ((A) to (B) of FIG. 9). At the central portion of the blood vessel, the smooth muscle and fibroblast cells are small in number and a fibrous material is rich ((C) of FIG. 9).

### Example 3

### Hardness Control of the Stenotic Matter at Totally Occluded Area

The stent made in Example 1 and coated with a 0.1 μm thick parylene C deposition film was crimped on a PTCA balloon and sterilized with an ethylene oxide gas. This was indwelled in the anterior descending coronary of the left coronary artery of a pig (35 to 45 kg) under anesthesia at a dilation ratio being 1.3 times greater. The pig was orally administered with antiplatelet drugs (330 mg of aspirin and 200 mg of ticlopidine) and a beta-blocking drug (10 mg of propranolol hydrochloride) from three days before the indwelling till the day before autopsy.

Angiography was carried out at the second or fourth week after the above treatment, confirming that the intended blood vessel was totally occluded.

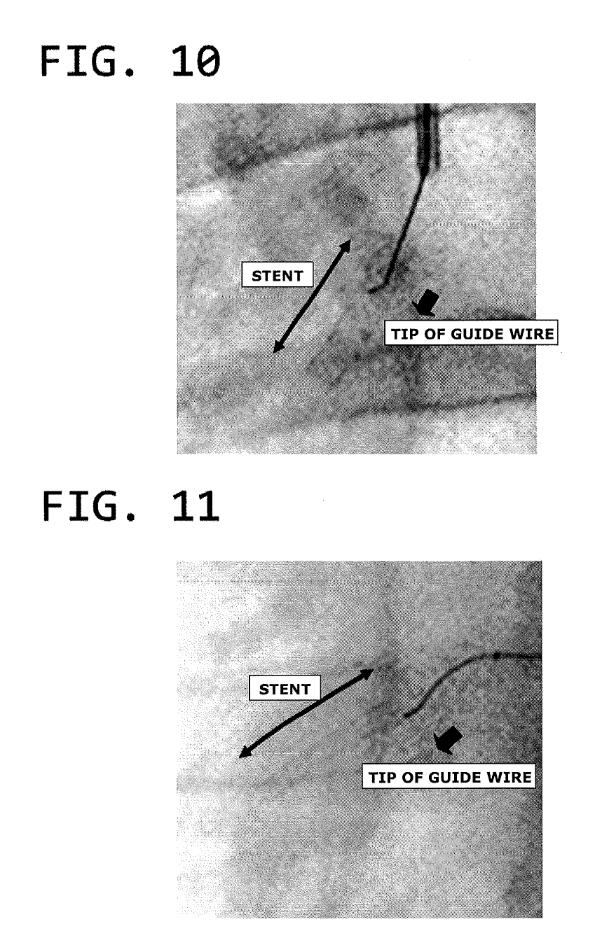

A guide wire was inserted from a guiding catheter, located at the opening of the left coronary artery, into the left anterior descending coronary artery and the tip of the guide wire was positioned short of the total occlusion area. Next, a microcatheter moved toward a tip direction along the guide wire and the tip of the microcatheter was closed to a tip portion of the guide wire. The tip of the guide wire was positioned at a recess of the total occlusion area, under which while rotating the guide wire, it was slowly moved inside the total occlusion area. Eventually, with the stent two weeks after the indwelling, the guide wire intruded into the total occlusion area (FIG. 10) but with a slight sense of resistance, whereas with the stent four weeks after the indwelling, the guide wire could not be moved into the total occlusion area (FIG. 11).

### Example 4

### Inspection of a Procedural Model Simulating the Therapy of Clinical Chronic Total Occlusion

A stent was coated with a parylene N deposited film in the same manner as in Example 1 except that parylene C was replaced by parylene N (Daisan Kasei Co., Ltd.) and the film thickness was changed to 0.01 μm.

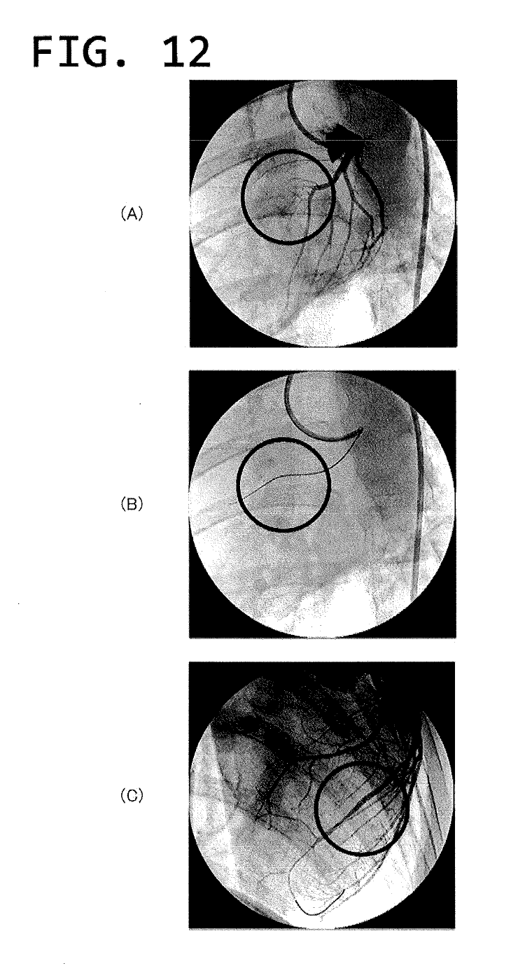

This stent was crimped on a PTCA balloon and sterilized with an ethylene oxide gas. This was indwelled in the anterior descending coronary of the left coronary artery of a pig (35 to 45 kg) under anesthesia at a dilation ratio being 1.3 times greater. The pig was orally administered with antiplatelet drugs (330 mg of aspirin and 200 mg of ticlopidine) and a beta-blocking drug (10 mg of propranolol hydrochloride) from three days before the indwelling till the day before autopsy. Angiography was carried out at the twelfth day after the above therapy, confirming that the intended blood vessel was totally occluded (FIG. 12(A)).

The following procedure simulating the therapy of clinical CTO was carried out. Initially, a guide wire was inserted from a guiding catheter, located at the opening of the left coronary artery, into the left anterior descending coronary artery and the tip of the guide wire was positioned short of the total occlusion area. Next, a microcatheter moved toward a tip direction along the guide wire and the tip of the microcatheter was closed to a tip portion of the guide wire. The tip of the guide wire was positioned at a recess of the total occlusion area, under which while rotating the guide wire, it was slowly moved inside the total occlusion area and passed therethrough. While keeping the indwelling of the guide wire, the microcatheter was removed and, instead, a balloon catheter was thrust from the base end side of the guide wire and the tip balloon of the balloon catheter was stopped at a position within the total occlusion area (FIG. 12(B)).

The balloon was dilated to expand the total occlusion area. According to the above operations, the restart of the blood flow in the intended blood vessel that had been totally occluded was confirmed (FIG. 12(C)).

From the above results, there was verified an establishment as the procedural model simulating the clinical therapy of the chronic total occlusion.

### Example 5

### Cardiac Infarction Model

The stent made in Example 1 and coated with a 0.1 μm thick parylene C deposition film was crimped on a PTCA balloon, sterilized with an ethylene oxide gas, and indwelled at the upstream or downstream side of the first diagonal branch of the left anterior descending coronary artery of a pig (35 to 40 kg) under anesthesia at a dilation ratio being 1.3 times greater. The pig was orally administered with antiplatelet drugs (330 mg of aspirin and 200 mg of ticlopidine) and a beta-blocking drug (10 mg of propranolol hydrochloride) from three days before the indwelling till the day before autopsy. Angiography was carried out at the fourteenth day after the above treatment, confirming that the intended blood vessel was totally occluded.

The echocardiographic examination revealed that the left ventricular ejection fraction (EF) lowered to 22 to 25% and the fractional area change (FAC) of the left ventricular cavity lowered to 30 to 36%, resulting in the significant deterioration of heart function.

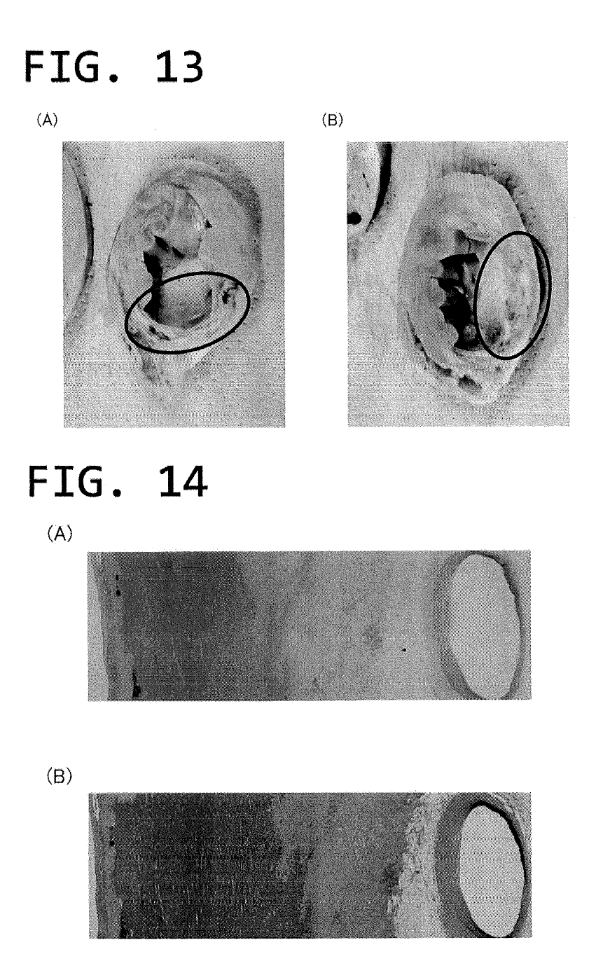

A cross-sectional observation image of the infarction area is shown in FIG. 13 and the pathological image is shown in FIG. 14. Visual observation after the treatment of euthanasia demonstrated that infarction was recognized around the anterior wall of heart in case where the stent was indwelled upstream of the first diagonal branch (FIG. 13(B)) and was recognized from the anterior wall to the ventricular septum in case where the stent was indwelled downstream of the first diagonal branch (FIG. 13(A)). The cardiac muscle of the infarction area was cut off to prepare a paraffin-embedded section, followed by HE staining (FIG. 14(A)) and Masson trichrome staining (FIG. 14(B)). The necrosis of the cardiac muscle and the infiltration and fibrosis of neutrophils were recognized around the blood vessel, and the degeneration region covered a wide range and inflammation was not pronounced, for which it was verified that the region where the flow of blood was interrupted due to the coronary occlusion without direct change thereof caused by the elution of metal ions.

From the above, it was verified that an ischemic myocardial model could be made by use of the indwelling vascular device of the invention and that an infarct area could be controlled.

### Comparative Example 1

### Stenosis with Sus 316L Stent

A coronary artery stent (φ3.0 mm×length 15 mm) made only of SUS 316L was crimped on a PTCA balloon and subsequently sterilized with an ethylene oxide gas. Six indwelling cases were carried out in such a way that the stent was indwelled in the left anterior descending coronary artery or the right coronary artery of a pig (35 to 40 kg) under anesthesia at a dilation ratio being 1.3 times greater. The pig was orally administered with antiplatelet drugs (330 mg of aspirin and 200 mg of ticlopidine) from three days before the indwelling till the day before autopsy.

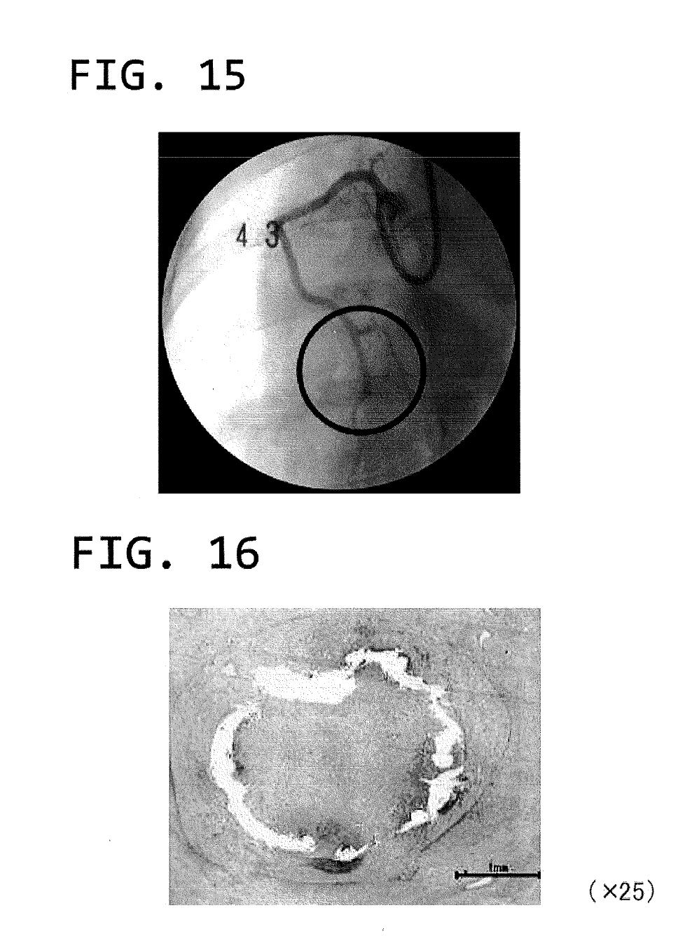

Angiography was carried out at the fourth week after the above treatment, with the result that % DS was at 2.1% (FIG. 15).

From the above results, evidence was given that when no elutable copper ions exist, not only does stenosis not proceed, but so too total occlusion is not reached.

### Comparative Example 2

### Adaptation of Copper-Plated Stent Having No Parylene Film

The coronary artery stent (p 3.0 mm×length 15 mm) made of SUS 316L and described in Comparative Example 1 was subjected to copper plating in a thickness of 5 μm, followed by crimping on a PTCA balloon and sterilization with an ethylene oxide gas. This was indwelled in the left anterior descending coronary artery of five pigs (40 to 45 kg) under anesthesia at a dilation ratio being 1.3 times greater. The pigs were each orally administered with antiplatelet drugs (330 mg of aspirin and 200 mg of ticlopidine) and a beta-blocking drug (10 mg of propranolol hydrochloride) from three days before the indwelling till the day before autopsy.

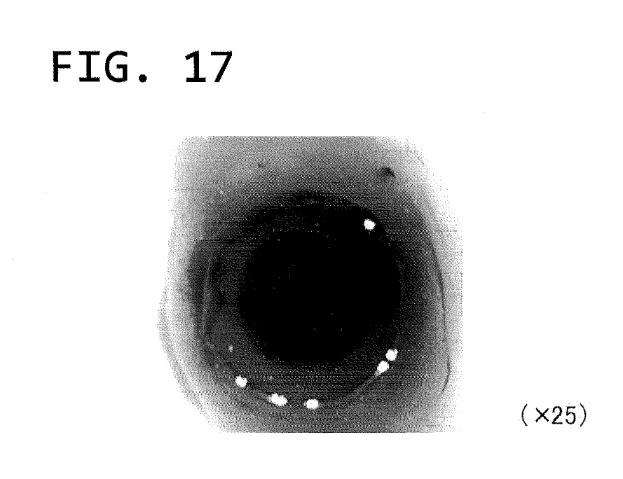

One pig died three hours after the indwelling and two further pigs were dead the next day. These dead cases were immediately subjected to autopsy, from which the death was judged to result from the acute coronary occlusion ascribed to the blood clot at the stent-indwelled area (FIG. 16). The other two survived for two weeks (survival rate 40%). According to angiography, total occlusion was confirmed, and a number of inflammatory cells and smooth muscle and fibroblast cells were observed as a result of the pathological observation of the blood vessel taken out from the euthanized animals (FIG. 17).

From the above, where no parylene film is deposited, acute thrombosis is brought about and the lethal rate is as high as 60%, for which not only the use as a model needs a great costly load and is not versatile, but also an objective, stable and accurate assessment is impossible, giving evidence of the effect of parylene.

The detailed description above describes features and aspects of embodiments of an indwelling vascular device for making a vascular stenosis model in animals, a stenosis or total occlusion model of a blood vessel in animals using the same, and a method of making the same, which are disclosed by way of example. The invention is not limited, however, to the precise embodiments and variations described. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. An indwelling vascular device comprising a device substrate sized for placement in a blood vessel and configured to ensure vascular flow immediately after indwelling in the blood vessel, the device substrate being made at least in part of a metal and/or metal compound which elutes toxic copper metal ions, and a polymer coating layer applied to device substrate on at least a surface of the device substrate containing the metal and/or metal compound which elutes toxic copper metal ions.

2. The indwelling vascular device according to claim 1, wherein the device substrate is fabricated entirely of the metal and/or metal compound which elutes toxic copper metal ions.

3. The indwelling vascular device according to claim 1, wherein the metal and/or metal compound which elutes toxic copper metal ions is a surface portion of the device substrate.

4. An indwelling vascular device comprising a device substrate containing a metal and/or metal compound which elutes toxic metal ions and having a structure ensuring a vascular flow immediately after indwelling in a blood vessel, and a polymer coating layer formed on at least a surface of the device substrate containing the metal and/or metal compound.

5. The indwelling vascular device according to claim 4, wherein the metal and/or metal compound is contained in a surface portion of said substrate.

6. The indwelling vascular device according to claim 4, wherein the polymer contains units derived from para-xylylene and/or a derivative thereof.

7. The indwelling vascular device according to claim 4, wherein the polymer coating layer possesses a thickness not less than 0.01 μm.

8. The indwelling vascular device according to claim 4, wherein the polymer coating layer possesses a thickness not greater than 3 μm.

9. The indwelling vascular device according to any of claim 4, wherein the toxic metal ions consist essentially of copper ions.

10. The indwelling vascular device according to claim 9, wherein an elution amount of copper per unit device ranges 4 to 1500 μl when measured according to JIS T0304.

11. The indwelling vascular device according to any of claim 4, wherein said device is a stent.

12. A method for making a vascular stenosis model, comprising indwelling the indwelling vascular device set forth in claim 4 in a blood vessel of a non-human healthy animal.

13. The method for making a vascular stenosis model according to claim 13, comprising controlling a stenosis rate by selecting a film thickness of the polymer coating layer of the vascular indwelling device.

14. A vascular stenosis model for non-human animals comprising an indwelling vascular device as set forth in claim 4, indwelled in a blood vessel.

15. The vascular stenosis model for non-human animals according to claim 14, wherein the blood vessel in which the indwelling vascular device is indwelled is completely occluded.

16. The vascular stenosis model for non-human animals according to claim 14, wherein the blood vessel in which the indwelling vascular device is indwelled is a coronary artery.

17. The vascular stenosis model for non-human animals according to claim 14, wherein the animal in which the indwelling vascular device is indwelled is a pig.

18. The vascular stenosis model for non-human animals according to claim 14, wherein the model serves for evaluation of a cardiopulmonary function or as a cardiopulmonary observation model in a clinical condition of vascular stenosis or total occlusion.

19. The vascular stenosis model for non-human animals according to claim 14, wherein the model is a training model for a guide wire procedure.

20. The vascular stenosis model for non-human animals according to claim 14, wherein the model is a regeneration therapy model for cardiac infarct.
